(19) United States
(12) Patent Application Publication
(10) Pub. No.: US 2020/0064356 A1
(43) Pub. Date: Feb. 27, 2020

(54) METHOD FOR DIAGNOSING AND TREATING ATTENTION DEFICIT HYPERACTIVITY DISORDER

(71) Applicant: EpicGenetics, Inc., Los Angeles, CA (US)

(72) Inventor: Bruce S. GILLIS, Beverly Hills, CA (US)

(21) Appl. No.: 16/543,741

(22) Filed: Aug. 19, 2019

Related U.S. Application Data
(60) Provisional application No. 62/720,319, filed on Aug. 21, 2018.

(51) Int. Cl. G01N 33/68; G01N 33/547

(52) U.S. Cl. CPC G01N 33/6863; C07K 16/246; G01N 33/547

(57) ABSTRACT

The invention provides methods and compositions for treating attention-deficit/hyperactivity disorder (ADHD) in an individual. The methods provided herein entail administering a composition comprising an isolated Mycobacterium or antigenic fragments derived therefrom. Also provided herein are methods for assessing alleviation of symptoms and/or alteration of immune system functioning following administration of a composition comprising an isolated Mycobacterium or antigenic fragments derived therefrom.

### CROSS-REFERENCE TO RELATED APPLICATIONS

This application claims the benefit of priority to U.S. Provisional Application No. 62/720,319, filed Aug. 21, 2018, which is herein incorporated by reference in its entirety for all purposes.

### FIELD OF THE INVENTION

This invention provides methods for treating attention-deficit/hyperactivity disorder (ADHD) using compositions comprising isolated Mycobacteria or antigenic fragments derived therefrom. Provided herein are also methods for diagnosing ADHD by measuring levels of one or more select cytokines.

### BACKGROUND OF THE INVENTION

Attention-deficit/hyperactivity disorder (ADHD) is a brain disorder marked by an ongoing pattern of inattention and/or hyperactivity-impulsivity that interferes with functioning or development. Some people with ADHD only have problems with one of the behaviors, while others have both inattention and hyperactivity-impulsivity. While the etiology of ADHD is not currently understood, there are a number of risk factors that can contribute to ADHD such as genes, cigarette smoking during pregnancy, alcohol use during pregnancy, drug use during pregnancy, exposure to environmental toxins such as high levels of lead at a young age, low birth weight and/or brain injuries. Further, ADHD is more common in males than females and females with ADHD are more likely to have problems primarily with inattention. Additionally, other conditions such as learning disabilities, anxiety disorder, conduct disorder, depression, and substance abuse are common in people with ADHD.

Currently, diagnosis of ADHD requires a comprehensive evaluation by a licensed clinician, such as a pediatrician, psychologist or psychiatrist with expertise in ADHD. For a person to receive a diagnosis of ADHD, the symptoms of inattention and/or hyperactivity-impulsivity must be long-lasting, impair the person's functioning and cause the person to fall behind normal development for his or her age and other medical or psychiatric conditions that manifest in similar ADHD-like symptoms need to be ruled out. Most children with ADHD receive a diagnosis during the elementary school years, while an adolescent or adult will only receive an ADHD diagnosis if the symptoms have been present since before age 12.

While there is no cure for ADHD, currently available treatments can help reduce symptoms and improve functioning. Treatments include medication, psychotherapy, education or training, or a combination of treatments. Current medications are geared toward reducing hyperactivity and impulsivity and improving a patient's ability to focus, work and learn and can include stimulants, non-stimulants and/or anti-depressants. Psychotherapy techniques can help patients and their families to better cope with everyday problems and can include behavioral therapy, cognitive behavioral therapy, and/or family and marital therapy. Additionally, stress management techniques can benefit parents of children with ADHD by increasing their ability to deal with frustration so that they can respond calmly to their child's behavior, while support groups can help parents and families connect with others who have similar problems and concerns.

Accordingly, there is a need for alternative methods of diagnosing and treating ADHD that have long lasting effects on a majority or all of the symptoms associated with ADHD. The present invention addresses this and other needs.

### SUMMARY OF THE INVENTION

In one aspect, provided herein is a method of treating attention-deficit/hyperactivity disorder (ADHD) and the symptoms associated with such a disorder in a subject, comprising administering a therapeutically effective amount of a composition comprising an isolated Mycobacterium to the subject, wherein the subject suffers from or is suspected of suffering from attention-deficit/hyperactivity disorder (ADHD). In some cases, the method further comprises diagnosing the subject with ADHD prior to the administration of the therapeutically effective amount of the composition comprising an isolated Mycobacterium. In some cases, the diagnosing comprises testing of the subject for an allergic disorder. In some cases, the allergic disorder is selected from asthma, eczema, rhinitis, urticaria or any combination thereof. In some cases, the diagnosing comprises conducting an assessment of immunologic traits associated with ADHD, an assessment of behaviors associated with ADHD or any combination thereof. In some cases, the immunologic traits are alterations in chemokine and/or cytokine production, wherein the alterations are an increase in production of one or more chemokines or cytokines, a decrease in production in one or more chemokines or cytokines or any combination thereof. In some cases, the immunologic traits associated with ADHD are occurrence of allergic disorders, atopy, hypersensitivity to allergens or any combination thereof. In some cases, the allergic disorders are asthma, eczema, rhinitis, urticaria, or any combination thereof. In some cases, the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample. In some cases, the blood sample obtained from the subject is peripheral blood mononuclear cells (PBMCs) isolated from plasma of the blood sample obtained from the subject. In some cases, the PBMCs are stimulated with one or more mitogens prior to determination of the expression levels of the at least four cytokines. In some cases, the determined expression level is a protein expression level, wherein the protein expression level is determined using an antibody bead-based capture assay that comprises beads derivatized with antibodies specific to the at least four cytokines. In some cases, the Mycobacterium comprises a whole cell Mycobacterium. In some cases, the Mycobacterium comprises a live-attenuated Mycobacterium. In some cases, the Mycobacterium comprises a heat-killed Mycobacterium. In some cases, the Mycobacterium is M. bovis. In some cases, the Mycobacterium is a Bacille Calmette-Guerin (BCG) strain. In some cases, the heat-killed Mycobacterium is a non-pathogenic Mycobacterium. In some cases, the non-pathogenic Mycobacterium is selected from M. vaccae, M. obuense, M. parafortunum, M. aurum, M. indicus pranii, and combinations thereof. In some cases, the Mycobacterium is selected from M. vaccae or M. obuense. In some cases, the Mycobacterium is a rough variant. In some cases, the Mycobacterium is in the form of a vaccine composition optionally comprising an adjuvant. In some cases, the vaccine composition induces one or more epigenetic changes in the genome of the individual. In some cases, the Mycobacterium is administered in repeat doses. In some cases, the Mycobacterium is administered in a unit dose comprising an effective amount of Mycobacterium from 10⁷ to 10⁹ cells. In some cases, the Mycobacterium is administered in a unit dose comprising an effective amount of Mycobacterium from 0.1 mg to 1 mg. In some cases, the Mycobacterium is formulated for administration via the parenteral, oral, sublingual, nasal or pulmonary route. In some cases, the Mycobacterium is formulated for administration via the oral route. In some cases, the parenteral route is selected from subcutaneous, intradermal, subdermal, intraperitoneal, intravenous, or intravesicular injection. In some cases, the administration of the composition comprising the isolated Mycobacterium prevents, reduces or alleviates at least one sign or symptom of ADHD, wherein the sign or symptom is selected from inattention, impulsivity, hyperactivity or a combination thereof. In some cases, the administration of the composition comprising the isolated Mycobacterium elevates or increases immune system activity of the subject. In some cases, the elevation or increase in immune system function is evidenced by the production of T_{H}1 cytokines, upregulation of granzyme B or both.

In another aspect, provided herein is a method of elevating or increasing a subject's immune system function comprising administering a therapeutically effective amount of a Mycobacterium vaccine to the subject, wherein the subject suffers from or is suspected of suffering from attention-deficit/hyperactivity disorder (ADHD). In some cases, the method further comprises diagnosing the subject with ADHD prior to the administration of the therapeutically effective amount of the composition comprising an isolated Mycobacterium. In some cases, the diagnosing comprises testing of the subject for an allergic disorder. In some cases, the allergic disorder is selected from asthma, eczema, rhinitis, urticaria or any combination thereof. In some cases, the diagnosing comprises conducting an assessment of immunologic traits associated with ADHD, an assessment of behaviors associated with ADHD or any combination thereof. In some cases, the immunologic traits are alterations in chemokine and/or cytokine production, wherein the alterations are an increase in production of one or more chemokines or cytokines, a decrease in production in one or more chemokines or cytokines or any combination thereof. In some cases, the immunologic traits associated with ADHD are occurrence of allergic disorders, atopy, hypersensitivity to allergens or any combination thereof. In some cases, the allergic disorders are asthma, eczema, rhinitis, urticaria, or any combination thereof. In some cases, the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample. In some cases, the blood sample obtained from the subject is peripheral blood mononuclear cells (PBMCs) isolated from plasma of the blood sample obtained from the subject. In some cases, the PBMCs are stimulated with one or more mitogens prior to determination of the expression levels of the at least four cytokines. In some cases, the determined expression level is a protein expression level, wherein the protein expression level is determined using an antibody bead-based capture assay that comprises beads derivatized with antibodies specific to the at least four cytokines. In some cases, the elevation or increase in immune system function is evidenced by the production of T_{H}1 cytokines, upregulation of granzyme B or both. In some cases, the elevation or increase in immune system function is evidenced by a reduction or alleviation of at least one sign or symptom of ADHD. In some cases, the sign or symptom is selected from inattention, impulsivity, hyperactivity or a combination thereof. In some cases, the Mycobacterium vaccine induces one or more epigenetic changes in the genome of the individual. In some cases, the Mycobacterium vaccine comprises a live-attenuated Mycobacterium. In some cases, the Mycobacterium vaccine comprises a heat-killed Mycobacterium. In some cases, the Mycobacterium is M. bovis. In some cases, the Mycobacterium is a Bacille Calmette-Guerin (BCG) strain. In some cases, the heat-killed Mycobacterium is a non-pathogenic Mycobacterium. In some cases, the non-pathogenic Mycobacterium is selected from M. vaccae, M. obuense, M. parafortunum, M. aurum, M. indicus pranii, and combinations thereof. In some cases, the Mycobacterium is selected from M. vaccae or M. obuense. In some cases, the Mycobacterium is a rough variant. In some cases, the vaccine further comprises an adjuvant. In some cases, the Mycobacterium vaccine is administered in repeat doses. In some cases, the Mycobacterium vaccine is administered in a unit dose comprising an effective amount of Mycobacterium from 10⁷ to 10⁹ cells. In some cases, the Mycobacterium vaccine is administered in a unit dose comprising an effective amount of Mycobacterium from 0.1 mg to 1 mg. In some cases, the Mycobacterium vaccine is formulated for administration via the parenteral, oral, sublingual, nasal or pulmonary route. In some cases, the Mycobacterium vaccine is formulated for administration via the oral route. In some cases, the parenteral route is selected from subcutaneous, intradermal, subdermal, intraperitoneal, intravenous, or intravesicular injection.

In yet another aspect, provided herein is a method of treating attention-deficit/hyperactivity disorder (ADHD) and the symptoms associated with such a disorder in a subject, comprising diagnosing a subject with ADHD; and administering a therapeutically effective amount of a composition comprising an isolated Mycobacterium to the subject diagnosed with ADHD. In some cases, the diagnosing comprises testing of the subject for an allergic disorder. In some cases, the allergic disorder is selected from asthma, eczema, rhinitis, urticaria or any combination thereof. In some cases, the diagnosing comprises conducting an assessment of immunologic traits associated with ADHD, an assessment of behaviors associated with ADHD or any combination thereof. In some cases, the immunologic traits are alterations in chemokine and/or cytokine production, wherein the alterations are an increase in production of one or more chemokines or cytokines, a decrease in production in one or more chemokines or cytokines or any combination thereof. In some cases, the immunologic traits associated with ADHD are occurrence of allergic disorders, atopy, hypersensitivity to allergens or any combination thereof. In some cases, the allergic disorders are asthma, eczema, rhinitis, urticaria, or any combination thereof. In some cases, the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample. In some cases, the blood sample obtained from the subject is peripheral blood mononuclear cells (PBMCs) isolated from plasma of the blood sample obtained from the subject. In some cases, the PBMCs are stimulated with one or more mitogens prior to determination of the expression levels of the at least four cytokines. In some cases, the determined expression level is a protein expression level, wherein the protein expression level is determined using an antibody bead-based capture assay that comprises beads derivatized with antibodies specific to the at least four cytokines. In some cases, the Mycobacterium comprises a whole cell Mycobacterium. In some cases, the Mycobacterium comprises a live-attenuated Mycobacterium. In some cases, the Mycobacterium comprises a heat-killed Mycobacterium. In some cases, the Mycobacterium is M. bovis. In some cases, the Mycobacterium is a Bacille Calmette-Guerin (BCG) strain. In some cases, the heat-killed Mycobacterium is a non-pathogenic Mycobacterium. In some cases, the non-pathogenic Mycobacterium is selected from M. vaccae, M. obuense, M. parafortunum, M. aurum, M. indicus pranii, and combinations thereof. In some cases, the Mycobacterium is selected from M. vaccae or M. obuense. In some cases, the Mycobacterium is a rough variant. In some cases, the Mycobacterium is in the form of a vaccine composition optionally comprising an adjuvant. In some cases, the vaccine composition induces one or more epigenetic changes in the genome of the individual. In some cases, the Mycobacterium is administered in repeat doses. In some cases, the Mycobacterium is administered in a unit dose comprising an effective amount of Mycobacterium from 10⁷ to 10⁹ cells. In some cases, the Mycobacterium is administered in a unit dose comprising an effective amount of Mycobacterium from 0.1 mg to 1 mg. In some cases, the Mycobacterium is formulated for administration via the parenteral, oral, sublingual, nasal or pulmonary route. In some cases, the Mycobacterium is formulated for administration via the oral route. In some cases, the parenteral route is selected from subcutaneous, intradermal, subdermal, intraperitoneal, intravenous, or intravesicular injection. In some cases, the administration of the composition comprising the isolated Mycobacterium prevents, reduces or alleviates at least one sign or symptom of ADHD, wherein the sign or symptom is selected from inattention, impulsivity, hyperactivity or a combination thereof. In some cases, the administration of the composition comprising the isolated Mycobacterium elevates or increases immune system activity of the subject. In some cases, the elevation or increase in immune system function is evidenced by the production of T_{H}1 cytokines, upregulation of granzyme B or both.

### DETAILED DESCRIPTION OF THE INVENTION

### Definitions

Unless otherwise defined, all technical terms used herein have the same meaning as commonly understood by one of ordinary skill in the art to which this invention belongs.

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, “protein” and “polypeptide” are used synonymously to mean any peptide-linked chain of amino acids, regardless of length or post-translational modification, e.g., glycosylation or phosphorylation.

As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

As used herein, the term “at least a portion” or “fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

For PCR amplifications of the polynucleotides disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al. (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition WT vs. G/C content) of primer. A pair of bi-directional primers consists of one forward and one reverse primer as commonly used in the art of DNA amplification such as in PCR amplification.

The term “cytokine” as used herein refers to small proteins that are secreted by specific cells of the immune system and glial cells, and include lymphokines, interleukins, and chemokines and their corresponding receptors, such as but not limited to IL-1, IL-2, IL-4, IL-5, IL-6, IL-7, IL-8, IL-10, IL-12, IL-13, IL-15, IL-17, IL-21, IFN-γ, IFN-α, TNF-α, IP-10, MCP-1, MIG, MIP-α, MIP-β, GM-CSF, Eotaxin, RANTES, etc. In another aspect, the invention further includes determining the levels of one or more of IL-1RA, IL2R, IL-7, IL-12 (p40/p70), IL-13, IL-15, IL-17, IFN-α, IP-10, MIG, VEGF, G-CSF, EGF, FGF-basic and HGF. In yet another aspect, the invention also includes determining the levels of IL-9 and PDGF-BB or a combination thereof. The cytokine may be inflammatory or anti-inflammatory. In one embodiment, the cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of cytokine assayed maybe a mRNA, miRNA, or DNA.

As used herein, the term “attention-deficit/hyperactivity disorder (ADHD)” and “attention deficit disorder (ADD)” can be used interchangeably. In some cases, the term ADHD can be defined as found in the Diagnostic and Statistical Manual (DSM) such as, for example, DSM-5. In some cases, the term ADHD can refer to any of three subtypes: predominantly inattentive presentation, predominantly hyperactive/impulsive presentation, and combined presentation. In some cases, ADD can refer to ADHD of the inattentive presentation.

As used herein, the term “inattention” as applied to an individual or subject or patient can be defined as a trait wherein said individual, patient or subject can procrastinate, not complete tasks and/or frequently move from one uncompleted activity to another. Inattention can also refer to being disorganized, lacking focus, having a hard time paying attention to details and a tendency to make careless mistakes, having trouble staying on topic while talking, not listening to others, not following social rules, being forgetful about daily activities or being easily distracted by things like trivial noises or events that are usually ignored by others or any combination thereof.

As used herein, the term “hyperactivity” as applied to an individual or subject or patient can be defined as a trait wherein said individual, patient or subject can fidget and squirm when seated, get up frequently to walk or run around, run or climb a lot at inappropriate times, have trouble playing quietly or doing quiet hobbies, always be ‘on the go’, talk excessively or any combination thereof. Individuals, subjects or patients experiencing hyperactivity may show up as feelings of restlessness.

As used herein, the term “impulsivity” as applied to an individual or subject or patient can be defined as a trait wherein said individual, patient or subject can be inpatient, having a difficult time waiting to talk or react, having a hard time waiting their turn, blurt out answers before someone finishes asking them a question, frequently interrupt or intrude on others to an extent that social problems can arise, start conversations at inappropriate times, or any combination thereof.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient”, “subject” and “individual” are used interchangeably herein, and mean a mammalian subject to be treated, with human patients being preferred. In some cases, the methods of the invention find use in experimental animals, in veterinary applications, and in the development of animal models for disease, including, but not limited to, rodents including mice, rats, and hamsters, as well as primates.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is attention-deficit/hyperactivity disorder (ADHD), the result can be an alleviation of one or more symptoms of ADHD such as, for example, widespread pain. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

### Overview

The present invention provides compositions and methods for treating attention-deficit/hyperactivity disorder (ADHD) in an individual. In one embodiment, treating ADHD in an individual comprises administering a composition comprising a Mycobacterium or an antigenic fragment thereof to the individual. The Mycobacterium can be an isolated Mycobacterium or an antigenic fragment thereof. The isolated Mycobacterium or antigenic fragment thereof can be a vaccine. The vaccine can be any vaccine that induces epigenetic changes in an individual administered said vaccine. The epigenetic changes can serve to provide the individual with life-long immunity against re-occurrence of a disease or condition (e.g., ADHD). The epigenetic changes can be cis-acting or trans-acting. The epigenetic changes can include changes in DNA methylation and/or histone protein modification. In one embodiment, the Mycobacterium is a Bacille Calmette-Guerin (BCG) strain of Mycobacterium bovis (M. bovis). Further to this embodiment, the composition can be a BCG vaccine. The BCG vaccine can be any BCG vaccine known in the art and/or commercially available. The BCG vaccine can be live-attenuated or heat-killed. The BCG vaccine can comprise any BCG strain known in the art and/or provided herein. In one embodiment, the BCG vaccine comprises the Tokyo 172 strain of BCG (e.g., Type I or Type II). In one embodiment, the BCG vaccine comprises the Tice strain of BCG. In another embodiment, the Mycobacterium is a non-pathogenic Mycobacterium species such as, for example Mycobacterium vaccae or Mycobacterium obtuense. The non-pathogenic Mycobacterium can be live-attenuated or heat-killed. The individual may have been previously diagnosed with ADHD or may be suspected of suffering from or being afflicted with ADHD. In one embodiment, the individual was previously diagnosed as having ADHD using any diagnostic means or methods known in the art such as, for example, following a comprehensive evaluation by a licensed clinician, such as a pediatrician, psychologist or psychiatrist with expertise in ADHD. In one embodiment, the individual was previously diagnosed as having ADHD using any method or test known in the art that relies on identifying immunologic traits and/or behaviors associated with ADHD. The immunologic traits can be changes (e.g., increases, decreases or any combination thereof) of chemokine and/or cytokine production. In some cases, the test used to measure changes in chemokine/cytokine production can be the commercially available FM/a® Fibromyalgia test. In some cases, the test used to measure changes in chemokine/cytokine production can incorporate the methods disclosed in US20150301062A1, the contents of which are herein incorporated by reference in their entirety. In one embodiment, the individual was previously diagnosed as having ADHD through a combination of a comprehensive evaluation by a licensed clinician, such as a pediatrician, psychologist or psychiatrist with expertise in ADHD as well as an assessment of any other immunologic traits and/or behaviors associated with ADHD. The immunologic traits associated with ADHD can be occurrence of allergic disorders (e.g., asthma, eczema, rhinitis, and/or urticaria), atopy, hypersensitivity to allergens or any combination thereof. In some cases, the diagnosis of ADHD can rely, at least in part, on the evaluation or testing of the individual for an allergic disorder, such as, for example, allergic rhinitis. In another embodiment, the individual was previously diagnosed as having ADHD using the commercially available FM/a® Fibromyalgia test or utilizing the methods described in US20150301062A1 in combination with a comprehensive evaluation by a licensed clinician, such as a pediatrician, psychologist or psychiatrist with expertise in ADHD. In yet another embodiment, the individual was previously diagnosed as having ADHD using the commercially available FM/a® Fibromyalgia test or utilizing the methods described in US20150301062A1 in combination with assessing other immunologic traits and/or behaviors associated with ADHD.

In another embodiment, the ADHD diagnosis can be determined by detecting the levels of one or more chemokines or cytokines in a sample obtained from an individual to see if the levels of the one or more chemokines or cytokines are altered. In one embodiment, the method comprises determining whether the levels of at least two, at least three, at least four, at least five, at least six, at least seven, at least eight, at least nine, at least ten, at least eleven, at least twelve or at least thirteen chemokines or cytokines, or at least thirteen or more chemokines or cytokines in an individual are altered. In one embodiment, the methods for diagnosing or predicting ADHD in a patient involve determining or assaying the levels of at least one, at least two, at least three, at least four, at least five, at least six, at least seven, at least eight, at least nine, or at least ten or more chemokines or cytokines in the plasma of blood samples obtained from individuals suspected of being afflicted with ADHD or at risk for ADHD. In a further embodiment, the method involves determining or assaying the levels of at least one, at least two, at least three, at least four, at least five, at least six, at least seven, at least eight, at least nine, or at least ten or more chemokines or cytokines in the peripheral blood mononuclear cells (PBMCs) that have been separated from the plasma of blood samples obtained from the individuals. These levels are then analyzed to determine if the levels are altered. The alteration may be an increase or decrease in expression of a chemokine or cytokine. The alteration can be determined at the protein and/or mRNA level as provided herein. The one or more chemokines or cytokines can be selected from the chemokines/cytokines listed in Table 1.

In one embodiment, altered expression is determined by comparing the chemokine/cytokine levels of the individual's sample to control levels. Control levels, in one embodiment, are determined by testing a sample from a healthy individual or an individual known to not have ADHD. In another embodiment, control levels are known, for example, from a database. The altered level(s) of the chemokines/cytokines measured in the affected individual compared to the level from the control group is predictive/indicative of ADHD in the individual. In one embodiment, a positive diagnosis of ADHD is provided if at least about 33% of the chemokines/cytokines tested have altered expression, or at least about 33% or more of the chemokines/cytokines tested have altered expression. In another embodiment, a positive diagnosis of ADHD is provided if a majority of the chemokines/cytokines tested have altered expression. In a further embodiment, a positive diagnosis of ADHD is provided if at least about 67% of the chemokines/cytokines tested have altered expression, or at least about 67% or more of the chemokines/cytokines tested have altered expression. In a further embodiment, a positive diagnosis of ADHD is provided if at least about 75%, or at least about 75% or more of the chemokines/cytokines tested have altered expression. In even a further embodiment, a positive diagnosis of ADHD is provided if the expression level of every chemokine/cytokine tested, or about every chemokine/cytokine tested in the patient is altered. The sample can be a solid sample (e.g., tissue biopsy) or a liquid sample (e.g., blood sample or a fraction thereof). As described herein, altered expression can be an increase or decrease in expression. The altered expression can be at the protein and/or mRNA level as described herein.

The chemokine/cytokine levels in an individual with ADHD, for example, chemokine/cytokine levels in a ADHD patient's blood, in one embodiment, are higher than the chemokine/cytokine levels of a healthy patient, for each chemokine/cytokine tested. In another embodiment, the chemokine/cytokine levels in a ADHD patient's blood are lower than the chemokine/cytokine levels of a healthy patient, for each chemokine/cytokine tested. In yet another embodiment, the chemokine/cytokine levels measured in a patient with ADHD may be higher or lower, depending on the panel of chemokine/cytokines measured in the individual. The panel of chemokines/cytokines for use in the methods provided herein can be selected from the panels found in Table 1.

The present invention is not limited by any particular combination of chemokines/cytokines. For example, the expression levels of chemokines/cytokines included in commercial chemokine/cytokine panels (or chemokine/cytokine subsets thereof) can be evaluated by the methods provided herein. Various combinations of chemokines/cytokines for use in the present invention are provided in Table 1 below. Subsets of these combinations may also be used in the methods provided herein. It should be understood that these combinations are representative, and should not be construed as limiting the invention.

TABLE 1
Non-limiting cytokine panels for use with the present invention.

| Panel 1 | Panel 2 | Panel 3 | Panel 4 | Panel 5 |
|---|---|---|---|---|
| IL5 | IFN-γ | TranSignal | Bio-Plex Pro | five or more |
| IL6 | IL-1β | Human Cytokine | magnetic | chemokines |
| IL8 | IL-2 | Antibody Array | Cytokine Assay | |
| IL 10 | IL-4 | 3.0 | (any of these | |
| IFN-γ | IL-5 | (or a subset of | assays may be | |
| MCP-1 | IL-6 | cytokines | used, i.e., the 8- | |
| MIP-α | IL-8 | provided in this | plex, 17-plex, 21- | |
| MIP-β | IL-10 | assay) | plex, 27-plex | |
| | TNF-α | | | |
| | MIP-1β | | | |
| | MCP-1 | | | |
| | MIP-1α | | | |
| | Rantes | | | |

| Panel 6 | Panel 7 | Panel 8 | Panel 9 | Panel 10 |
|---|---|---|---|---|
| IL5 | IL-6 | IL-2 | IL-8 | IFN-γ |
| IL6 | IL-8 | IL-4 | IL-10 | IL-1β |
| IL8 | IL-10 | IL-5 | TNF-α | IL-2 |
| IL 10 | TNF-α | IL-6 | MIP-1β | |
| IFN-γ | MIP-1β | | MCP-1 | |
| | MCP-1 | | Rantes | |
| | MIP-1α | | | |

In one embodiment, administration, as defined herein, includes the administration of the Mycobacterium or an antigenic fragment thereof in multiple aliquots and/or doses and/or on separate occasions. The Mycobacterium or an antigenic fragment thereof can be present in a vaccine. The vaccine can include an adjuvant. The adjuvant can be any adjuvant known in the art and/or provided herein.

In one aspect of the present invention, the Mycobacterium comprises a live-attenuated strain of a Mycobacterial species or an antigenic fragment thereof. In another aspect of the present invention the Mycobacterium comprises a heat-killed strain of a Mycobacterial species or an antigenic fragment thereof. Mycobacterial species for use in the present invention include, but are not limited to M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. indicus pranii, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diemhoferi, M. smegmatis; M. thamnopheos; M. flavescens; M. fortuitum; M. peregrinum; M. chelonei; M. paratuberculosis; M. leprae; M. lepraemurium; M. bovis and combinations thereof.

In one embodiment, the Mycobacterial species is M. bovis. The M. bovis can be any known strain of M. bovis. In one embodiment, the M. bovis is a BCG strain. The BCG can be heat-killed or live-attenuated. The BCG can be any known BCG vaccine. The strain of BCG can be any of the strains belonging to BCG groups DU1, DU2-I, DU2-II, DU2-III and DU2-IV. The strain of BCG can be selected from BCG Pasteur (1961), BCG Moreau, BCG Russia (1924), BCG Japan (1925), BCG Tokyo 172 (Type I, Type II) or a combination thereof, BCG Sweden (1927), BCG Birkhaug, BCG Prague, BCG Glaxo (1954), BCG Merieux (1989), BCG Danish, BCG Frappier, BCG Connaught (1948), BCG Mexico, BCG Tice (1934), BCG China or BCG Phipps.

In one embodiment, the Mycobacterium for use in the methods and compositions (e.g., vaccine compositions) of the present invention is a Tokyo strain of BCG. In one embodiment, the Mycobacterium for use in the methods and compositions (e.g., vaccine compositions) of the present invention is BCG Tokyo 172 (ATCC 35737; TMC1019). The BCG Tokyo strain for use in the methods and compositions provided herein can be the Type I or Type II subpopulations or a combination or mixture thereof. It should be noted that the two subpopulations differ in their colony morphologies with Type I being smooth and Type II being rough. Further, smooth colonies have a characteristic 22-bp deletion in Rv3405c of the region of difference (RD) 16 (type I), while rough colonies are complete in this region (type II). Additionally, the subpopulation types also differ in their lipid phenotypes, with phenolic glycolipid (PGL) and phthiocerol dimycocerosate (PDIM) being found only in type I. Given that PGL has been shown to suppress the host recognition of total lipids via Toll-like receptor 2, it may be antigenic and involved in host responses, acting as a cell wall component.

In one embodiment, the strain of BCG present in a composition (e.g., vaccine composition) as provided herein is a genetically engineered strain of BCG. The strain of BCG can be genetically engineered to improve or enhance immune system function such as, for example, T cell-mediated immunity. The strain of BCG can be genetically engineered to be equipped with the membrane-perforating listeriolysin (Hly) of Listeria monocytogenes as shown in Grode et al. Increased vaccine efficacy against tuberculosis of recombinant Mycobacterium bovis bacille Calmette-Guérin mutants that secrete listeriolysin, J. of Clinical Investigation, Vol. 115: 9, September 2005, which is herein incorporated by reference in its entirety.

In one embodiment, the Mycobacterium is non-pathogenic. The non-pathogenic Mycobacterium can be heat-killed. The non-pathogenic Mycobacterium can be selected from M. vaccae, M. obuense, M. parafortuitum, M. aurum, M. indicus pranii, M. phlei and combinations thereof. In one embodiment, the non-pathogenic Mycobacterium is a rough variant. In one embodiment, the non-pathogenic Mycobacterium is a smooth variant. In one embodiment, the methods provided herein comprise administering a composition comprising M. vaccae. Further to this embodiment, the M. vaccae can be heat-inactivated. Further still to this embodiment, the M. vaccae can be strain NCTC11659. Even further still to this embodiment, the composition comprising M. vaccae is SRL172. SRL172 is a suspension of heat-killed whole cell Mycobacterium vaccae. In one embodiment, the methods provided herein comprise administering a composition comprising M. obuense. Further to this embodiment, the M. obuense can be heat-inactivated. Further still to this embodiment, the M. obuense can be strain NCTC13365. Even further still to this embodiment, the composition comprising M. obuense is IMM-101. IMM-101 is a suspension of heat-killed whole cell Mycobacterium obuense.

In one embodiment, the compositions provided herein can be used as vaccines and can accordingly be formulated as pharmaceutical compositions.

In one embodiment, administration of the composition comprising the isolated Mycobacterium or antigenic fragment thereof reduces, eliminates or alleviates one or more symptoms of ADHD. The one or more symptoms can be selected from hyperactivity, inattention, impulsivity or any combination thereof. The reduction, elimination or alleviation can be as compared to a control. The control can be the individual prior to administration of the composition comprising the isolated Mycobacterium or antigenic fragment thereof or a separate individual suffering from ADHD. In another embodiment, administration of the composition comprising the isolated Mycobacterium or antigenic fragment thereof increases immune system functioning. The increase in immune system functioning can be evidenced by the production or elevation thereof of T_{H}1 cytokines, upregulation of granzyme B or both. The T_{H}1 cytokines that are elevated or produced in response to administration of the compositions provided herein can include IFN-γ, IL-2, or TNF-β or a combination thereof.

### Administration of Compositions

In certain embodiments, the compositions described herein (e.g., the immunogenic compositions) comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an isolated Mycobacterium or antigenic fragment derived therefrom but when the compound is administered alone does not generate an immune response to the isolated Mycobacterium or antigenic fragment derived therefrom. In some embodiments, the adjuvant generates an immune response to the isolated Mycobacterium or antigenic fragment derived therefrom and does not produce an allergy or other adverse reaction. Adjuvants can enhance an immune response by several mechanisms including, e.g., lymphocyte recruitment, stimulation of B and/or T cells, and stimulation of macrophages. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants for use in the methods and compositions provided herein can include, but are not limited to, cytokines (e.g., IL-12), heat-shock proteins, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), ASO4 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, N Y, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)).

The compositions provided herein comprise the isolated Mycobacterium or antigenic fragments derived therefrom alone or, preferably, together with a pharmaceutically acceptable carrier. Suspensions or dispersions of isolated Mycobacterium or antigenic fragments derived therefrom, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. The said dispersions or suspensions may comprise viscosity-regulating agents. The suspensions or dispersions are kept at temperatures around 2-4° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprises 0.001% to 0.01% thimerosal. In other embodiments, the pharmaceutical compositions described herein do not comprise a preservative.

The compositions of the invention may be administered to mammals (e.g., rodents, humans) in any suitable formulation. For example, isolated Mycobacterium or antigenic fragments thereof may be formulated in pharmaceutically acceptable carriers or diluents such as physiological saline or a buffered salt solution. Suitable carriers and diluents as provided herein can be selected on the basis of mode and route of administration and standard pharmaceutical practice.

The compositions of the invention may be administered to mammals by any conventional technique. Typically, such administration will be oral, sublingual, nasal, pulmonary or parenteral (e.g., intravenous, subcutaneous, intravesicular, intramuscular, intraperitoneal, intradermal, subdermal, or intrathecal introduction). The compositions may also be administered directly to a target site by, for example, surgical delivery to an internal or external target site, or by catheter to a site accessible by a blood vessel. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Dosing

The compositions (e.g., vaccine compositions) described above are preferably administered to a mammal (e.g., a rat, human) in an effective amount, that is, an amount capable of producing a desirable result in a treated individual (e.g., activating or boosting the immune response). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

In certain embodiments, a particular dosage of a composition provided herein (e.g., vaccine comprising a strain of BCG) is administered to a subject. In certain embodiments of the invention, there is provided a composition comprising a live-attenuated or heat-killed bacteria (e.g., strain of BCG) for use in the present invention, which typically may be from 10³ to 10¹¹ cells or colony forming units (CFUs), from 10⁴ to 10¹⁰ cells or CFUs, from 10⁶ to 10¹⁰ cells or CFUs, or 10⁶ to 10⁹ cells or CFUs per unit dose. The effective amount of live-attenuated or heat-killed Mycobacterium for use in the methods or compositions provided herein can be from 10³ to 10¹¹ cells or CFUs, from 10⁴ to 10¹⁰ cells or CFUs, from 10⁶ to 10¹⁰ cells or CFUs, and from 10⁶ to 10⁹ cells or CFUs per unit dose. The unit dose can be 5 ul, 10 ul, 20 ul, 30 ul, 40 ul, 50 ul, 60 ul, 70 ul, 80 ul, 90 ul, 100 ul, 125 ul, 150 ul, 175 ul, 200 ul, 250 ul, 300 ul, 350 ul, 400 ul, 450 ul, 500 ul, 600 ul, 650 ul, 700 ul, 750 ul, 800 ul, 850 ul, 900 ul, 950 ul, 1000 ul or 1500 ul. In one embodiment, the composition comprises a therapeutically effective amount of live-attenuated or heat-killed Mycobacterium (e.g., strain of BCG such as Tokyo Strain) is from 1.8×10⁶ to 3.9×10⁶ colony forming units per unit dose, wherein the unit dose is 0.1 ml. Alternatively, the dose of a composition provided herein can be from 0.01 mg to 1 mg, 0.1 mg to 0.5 mg, 0.5 mg to 1 mg, 1 mg to 1.5 mg or 1.5 mg to 2.0 mg. In one embodiment, the dose is 1 mg. In one embodiment, the dose is 0.5 mg. In one embodiment, the dose is 0.1 mg. The organisms or antigenic fragments derived therefrom can be presented as either a suspension or dry preparation. Further to the above embodiments, the route of administration can be intradermal (ID) administration.

The preparation according to the intention may also comprise, such as pharmaceutically acceptable additives, e.g. solvents, adjuvants, carriers and/or preservatives as provided herein.

The methods of treatment for ADHD as provided herein is preferably conducted as a series of administrations with increasing doses during a specific period. In one example, the composition can be administered in 8-10 increasing doses during 4-12 weeks, preferably 8-10 weeks. The reason for the increasing doses can be that during the first week or weeks the patient may suffer from side effects, and it is therefore advantageously to start with a low dose. The side effects may diminish after some time. In one embodiment, the composition can be administered in 2 doses spaced 4 weeks apart.

In order to obtain the desired effect for a prolonged period of time the composition (e.g., vaccine comprising BCG) may be administered at several occasions. For example, a first series of administrations may be followed by repeated administrations given at specified intervals. The specified intervals can be approximately once a week for 5-15 weeks, preferably for 10 weeks.

To prevent recurrence, the repeated administrations may then be followed by a maintenance treatment with administrations at specified intervals. The specified intervals can be approximately once a month. The specified intervals may be continued for several years, such as 1-10 years, or approximately 5 years. In one embodiment, the maintenance treatment entails one injection of the composition per year for a specified interval of 4 years.

The doses in the repeated administrations of the maintenance treatment can be constant. In some cases, the doses in the maintenance treatment can be the dose used in the last administration in the first series.

These repeated administrations can result in an unspecific or specific activation of the immune system over a long period of time.

The administrations can be made in any way known in the art, such as, for example, injections.

Additional agents or substances can be administered simultaneously or in parallel with the vaccine compositions of the present invention.

### Assessing Treatment Efficacy

In another aspect, the invention provides methods for evaluating the efficacy of treatment in an individual diagnosed with ADHD. In one embodiment, ADHD treatment efficacy was assessed using any diagnostic means or methods known in the art and/or provided herein. For example, treatment efficacy can be assessed through the use of a comprehensive evaluation by a licensed clinician, such as a pediatrician, psychologist or psychiatrist with expertise in ADHD following treatment. In some cases, the results of the comprehensive evaluation post-treatment can be compared to the results of a comprehensive evaluation performed prior to treatment. In some cases, the results of the comprehensive evaluation post-treatment can be compared to the results of a comprehensive evaluation performed on a healthy individual. The healthy individual can be an individual not previously diagnosed or suspected of suffering from ADHD. In one embodiment, treatment efficacy on ADHD can be assessed by examining changes in immunologic traits and/or behaviors associated with ADHD following treatment (i.e., post-treatment). In some cases, the post-treatment results can be compared to an assessment of the immunologic traits and/or behaviors associated with ADHD prior to treatment (i.e., pre-treatment). In some cases, the post-treatment results can be compared to an assessment of the immunologic traits and/or behaviors associated with ADHD on a healthy individual. The healthy individual can be an individual not previously diagnosed or suspected of suffering from ADHD. The assessment of the immunologic traits and/or behaviors associated with ADHD can be determined using any method or test known in the art used to measure immunologic traits and/or behaviors associated with ADHD. In some cases, the test used to measure changes in chemokine/cytokine production can be the commercially available FM/a® Fibromyalgia test. In some cases, the test used to measure changes in chemokine/cytokine production can incorporate the methods disclosed in US20150301062A1, the contents of which are herein incorporated by reference in their entirety. In some cases, the test used to measure changes in chemokine/cytokine production can be measuring changes in one or more chemokines/cytokines found in Table 1. In some cases, the test used to measure changes in chemokine/cytokine production can be measuring changes in one or more panels of chemokines/cytokines found in Table 1. In one embodiment, treatment efficacy on ADHD can be assessed or determined through a combination of a comprehensive evaluation by a licensed clinician, such as a pediatrician, psychologist or psychiatrist with expertise in ADHD as well as an assessment of any other immunologic traits and/or behaviors associated with ADHD. The immunologic traits can be changes (e.g., increases, decreases or any combination thereof) of chemokine and/or cytokine production such as, for example, those found in Table 1. The immunologic traits associated with ADHD can be occurrence of allergic disorders (e.g., asthma, eczema, rhinitis, and/or urticaria), atopy, hypersensitivity to allergens or any combination thereof. In some cases, the treatment efficacy on ADHD can rely, at least in part, on the evaluation or testing of the individual for an allergic disorder, such as, for example, allergic rhinitis. In some cases, the methods of treatment provided herein can alleviate one or more symptoms associated with allergic disorder known or thought to be associated with or coincident with ADHD.

For example, the method involves determining or detecting as a baseline the level of one or more chemokines and/or cytokines expressed in the individual diagnosed with or suspecting of having ADHD prior to treatment. Following treatment, subsequent measurements of one or more cytokine levels are carried out to determine the levels or patterns of expression of the one or more cytokines. The altered levels and/or patterns of expression of one or more of the cytokines measured in the individual afflicted with ADHD or at risk for developing ADHD and undergoing treatment are compared to the levels or patterns of expression of cytokines in a control. In one embodiment, the control is the levels and/or patterns of expression of the one or more cytokines in the individual before treatment. In another embodiment, the control is the levels and/or expression levels of the one or more cytokines from a healthy patient, or cytokine levels reported for a patient without ADHD (for example, levels reported in a database). The one or more chemokines/cytokines can be selected from those found in Table 1.

In one embodiment, the methods for assessing treatment efficacy involve determining or assaying the levels of at least one, at least two, at least three, at least four, at least five, at least six, at least seven, at least eight, at least nine, or at least ten or more chemokines/cytokines in the plasma of blood samples obtained from individuals suspected of being afflicted with ADHD or at risk for ADHD after treatment with the compositions comprising isolated Mycobacterium or antigenic fragments thereof and comparing the levels of the assayed chemokines/cytokines to a control. The control can be any control as provided herein. In a further embodiment, the method involves determining or assaying the levels of at least one, at least two, at least three, at least four, at least five, at least six, at least seven, at least eight, at least nine, or at least ten or more chemokines/cytokines in the peripheral blood mononuclear cells (PBMCs) that have been separated from the plasma of blood samples obtained from the individuals after treatment with the compositions comprising isolated Mycobacterium or antigenic fragments thereof and comparing the levels of the assayed chemokines/cytokines to a control. The control can be any control as provided herein. These levels are then analyzed to determine if the levels are altered due to the treatment. For example, the levels in the individual's sample during and/or after treatment, in one embodiment, are compared to levels in a control sample, for example, a sample known to not have ADHD. In another embodiment, control levels are known, for example, from a database. In one embodiment, a change in expression in a majority of the chemokines/cytokines tested toward the levels in the control is determinative/indicative of the treatment for ADHD being efficacious. In another embodiment, a change in expression of at least about 33% or at least about 67% of the chemokines/cytokines tested is determinative/indicative of an effective treatment for ADHD. In a further embodiment, a treatment with a composition as provided herein is deemed to be efficacious for treating ADHD if at least about 75%, or at least about 75% or more of the chemokines/cytokines tested have altered expression due to the treatment. In even a further embodiment, a treatment with a composition as provided herein is deemed to be efficacious for treating ADHD if the expression level of every chemokine/cytokine tested, or about every chemokine/cytokine tested in the patient is altered. The altered expression of one or more chemokines/cytokines during or following treatment can be modifying the level or expression of the one or more chemokines/cytokines to be substantially the same expression level of the one or more chemokines/cytokines in a control (e.g., the levels in a healthy patient who does not have ADHD). As used herein, the term “substantially the same expression level” can be about 55%, about 60%, about 65%, about 70%, about 75%, about 80%, about 85%, about 90%, about 95%, about 99% or about 99% of the expression level of a particular chemokine/cytokine in a control as provided herein.

The present invention is not limited by any particular combination of chemokines/cytokines. For example, the chemokines/cytokines to whose expression can be evaluated in order to determine treatment efficacy can be selected from IL-1, IL-2, IL-4, IL-5, IL-6, IL-7, IL-8, IL-10, IL-12, IL-13, IL-15, IL-17, IL-21, IFN-γ, IFN-α, TNF-α, IP-10, MCP-1, MIG, MIP-α, MIP-β, GM-CSF, Eotaxin, RANTES, etc. or a combination thereof. In another aspect, the invention further includes determining the levels of one or more of IL-1RA, IL2R, IL-7, IL-12 (p40/p70), IL-13, IL-15, IL-17, IFN-α, IP-10, MIG, VEGF, G-CSF, EGF, granzyme B, FGF-basic and HGF or a combination thereof. In yet another aspect, the invention also includes determining the levels of IL-9 and PDGF-BB or a combination thereof. The chemokine/cytokine may be inflammatory or anti-inflammatory. In one embodiment, the chemokine/cytokine to be assayed may be a full length polypeptide, protein, a glycoprotein or a fragment thereof. Other proteins that can be assayed include hormones, heat-shock proteins, antibodies such as but not limited to anti-nuclear antibody (ANA), thyroid antibodies, anti-extractable nuclear antibodies (ENA), IgG subclasses, anti-nuclear factors (FAN), rheumatoid factor (RF), receptor proteins and ligands, etc. In other embodiment, the level of chemokine/cytokine assayed maybe a mRNA, miRNA, or DNA. In another example, the expression levels of chemokines/cytokines included in commercial chemokine/cytokine panels (or chemokine/cytokine subsets thereof) can be evaluated by the methods provided herein. Various combinations of cytokines for use in the present invention are provided in the Table 1 as provided herein. Subsets of these combinations may also be used in the methods provided herein. It should be understood that these combinations are representative, and should not be construed as limiting the invention.

### Measurement/Detection of Cytokine Levels

In one embodiment, chemokine/cytokine levels in methods entailing diagnosing and/or assessing treatment efficacy as provided herein are tested on the protein level. In another embodiment, chemokine/cytokine levels in methods entailing diagnosing and/or assessing treatment efficacy as provided herein are determined at the mRNA level. In yet another embodiment, both mRNA and protein levels for the chemokine/cytokines are examined in the methods provided herein. Methods for assaying chemokine/cytokines at the protein or mRNA levels are well known in the art and can be employed in the methods provided herein.

Measuring chemokine/cytokine levels in methods entailing diagnosing and/or assessing treatment efficacy as provided herein can be from blood or a plasma sample that may be stimulated or un-stimulated. That is, cell proliferation may be induced prior to assaying the chemokine/cytokine levels. In one embodiment, the PBMCs are un-stimulated. In another embodiment, the PBMCs are stimulated to cause proliferation of the cells prior to assaying for chemokine/cytokines. Methods for stimulating PBMCs are known in the art, and include, but are not limited to, the addition of mitogens to the cells. Non-limiting examples of mitogens include lipopolysaccharide (LPS), phytohemagglutinin (PHA), or phorbol ester, such as phorbol myristate acetate (PMA) with or without ionomycin, pokeweed mitogen (PWM), concavalin A (Con-A), or combinations thereof.

In one embodiment, chemokine/cytokine expression is measured at the mRNA level, for example, by quantitative RT-PCR (also known as real time RT-PCR). mRNA expression levels can also be measured by Northern blot assay, array hybridization, sequencing, etc. For example, multiplex quantitative RT-PCR, in one embodiment, is carried out to determine the mRNA expression levels of a chemokine/cytokine panel. Chemokine/Cytokine RT-PCR kits are commercially available, for example, from Roche.

In another embodiment, secreted chemokine/cytokine levels are determined (i.e., at the protein level). In one embodiment, secreted chemokine/cytokine levels are determined by using an antibody array, for example, the TranSignal Human Cytokine Antibody Array 3.0, available from Panomics. The Panomics array includes antibodies directed to the following cytokines: Apol/Fas, Leptin, Rantes, ICAM-1, IL-2, IL-7, CTLA, MIP1α, MIP1β, TGFβ, VCAM-1, IL-3, IL-8, IL-4, IL-10, IL-5, IL-12, IL-6, IL-15, IL-6R, IL-17, IL-1Rα, IL-1β, IL-1α, VEGF, IFNγ, TNFα, TNFRI, TNFRII, MIP-5, MIP4, MMP3, Eotaxin, GM-CSF, EGF, IP-10. In this embodiment, not all chemokine/cytokines in the array need be probed for. For example, the expression levels of a subset of five chemokine/cytokines, or five or more chemokine/cytokines, or six chemokine/cytokines, or six or more chemokine/cytokines, or seven chemokine/cytokines, or seven or more chemokine/cytokines, or ten chemokine/cytokines, or ten or more chemokine/cytokines, or twelve chemokine/cytokines, or twelve or more chemokine/cytokines may be determined when carrying out the methods of the invention.

Secreted chemokine/cytokine levels, in one embodiment, are determined with a multiplex immunoassay built on magnetic beads. For example, in one embodiment, the Bio-Plex Pro magnetic Cytokine Assay is used (Bio-Rad). In this embodiment, the Assay is commercially available as a ready to use kit, for example, for the detection of eight cytokines, seventeen cytokines, 21 cytokines or 27 cytokines. The full number or a subset of the cytokines may be detected in the methods of the invention. Alternatively, expression levels of cytokines can be tested in a sample by doing multiple assays on the sample, for example, in “singleplex” format. In one embodiment, the Bio-Rad singleplex cytokine assays are used.

Another antibody based bead assay is available from Invitrogen, and is also amenable to be used in the methods of the present invention. Specifically, the Human Cytokine Thirty-Plex antibody bead kit may be employed to detect the levels of a panel of cytokines in an individual. Although the assay can detect the levels of thirty cytokines, not all thirty need to be detected in order to carry out the methods provided herein. For example, as provided above, five, six, seven, eight, nine, ten, eleven or twelve cytokines can be assayed for their expression levels. The Invitrogen kit comprises analyte specific components for the measurement of human IL-1β, IL-1RA, IL-2, IL-2R, IL-4, IL-5, IL-6, IL-7, IL-8, IL-10, IL-12p40/p70, IL-13, IL-15, IL-17, TNF-α, IFN-α, IFN-γ, GM-CSF, MIP-1α, MIP-1β, IP-10, MIG, Eotaxin, RANTES, MCP-1, VEGF, G-CSF, EGF, FGF-basic, and HGF. These reagents, in one embodiment, are used in the Luminex® 100™ or 200™ System.

Methods for assaying chemokine/cytokines at the protein or mRNA levels are well known in the art. Besides the assays provided above, other non-limiting examples of methods for assaying chemokine/cytokines at the protein level include enzyme-linked immunoassay (ELISA), Tetramer assay, ELISPOT assay, Fluorospot assay, etc. The chemokine/cytokines concentration in the plasma, culture supernatant, or cell lysate derived from PBMC can be measured, for example, by multiplex immunoassay based on Luminex xMAP bead array technology, or Bio-Plex 200 fluorescence bead reader (BioRad Laboratories, Hercules, Calif.). In one embodiment, the level of one or more chemokine/cytokine mRNA can be detected (measured) by real time PCR, RT-PCR, Northern blot assay, array hybridization, sequencing, etc. The altered level(s) of the chemokine/cytokines measured in the affected individual compared to the level from control group is predictive/indicative of ADHD in the individual. The chemokine/cytokine levels in an individual with ADHD, for example, chemokine/cytokine levels in a ADHD patient's blood, in one embodiment, are higher than the chemokine/cytokine levels of a healthy patient, for each chemokine/cytokine tested. In another embodiment, the chemokine/cytokine levels in a ADHD patient's blood are lower than the chemokine/cytokine levels of a healthy patient, for each chemokine/cytokine tested. In yet another embodiment, the chemokine/cytokine levels measured in a patient with ADHD may be higher or lower, depending on the panel of chemokine/cytokines measured in the individual.

The level of chemokine/cytokines can be determined using an algorithm and the raw data obtained by measuring the levels of chemokine/cytokines which have been stored in a computer system, or any other medium that is linked to a computer or machine. In one aspect, the method further includes evaluation of the individual's clinical and physical symptoms in conjunction with determining the levels of one or more chemokine/cytokines. For example, the method includes evaluation of tender points in the individual. The pain threshold is calculated and assigned a score on a subjective basis. The scores derived from the assessment of the clinical and physical symptoms may be included in the statistical analysis for the chemokine/cytokines. In a further embodiment, the method includes determining the levels of various factors or markers, such as but not limited to Rheumatoid Factor (RF), or a specific marker of inflammation such as the erythrocyte sedimentation rate (ESR).

As it relates to the diagnostic and/or assessment of treatment efficacy methods provided herein, chemokine/cytokine expression can be “altered” or “differentially expressed”, in an individual, in one embodiment, if expression of the chemokine/cytokine in the individual's sample is at least about 1.5 times higher or lower than the expression of the same chemokine/cytokine at a control level. In another embodiment, chemokine/cytokine expression is “altered” if chemokine/cytokine expression in the individual's sample is at least about 2 times higher or lower than the expression of the same chemokine/cytokine at a control or baseline level (i.e., levels reported for a healthy patient). In another embodiment, chemokine/cytokine expression is “altered” if chemokine/cytokine expression in the individual is at least about 2.5 times higher or lower (or at least about 2.5 times or more higher or lower) than the control expression level of the same chemokine/cytokine. In yet another embodiment, chemokine/cytokine expression is “altered” if chemokine/cytokine expression in the individual is at least about 3 times higher or lower (or at least about 3 times or more higher or lower) than the control expression level of the same chemokine/cytokine. In another embodiment, chemokine/cytokine expression is “altered” if chemokine/cytokine expression in the individual is at least about 5 times higher or lower (or at least about 5 times or more higher or lower) than the control expression level of the same chemokine/cytokine. In even another embodiment, chemokine/cytokine expression is “altered” if chemokine/cytokine expression in the individual is at least about 10 times higher or lower than the control expression level of the same chemokine/cytokine in a control sample. In yet another embodiment, chemokine/cytokine expression is altered if chemokine/cytokine expression in the individual is at least about 10 times or more, higher or lower, than the control expression level of the same chemokine/cytokine. As provided above, control expression level may be determined from values in a database, from a non-disease sample (e.g., ADHD) or individual.

Altered expression of the chemokine/cytokine may be the same or different for each individual chemokine/cytokine that is differentially expressed. For example, the expression of one chemokine/cytokine (mRNA or protein) may be 2× lower, or about 2× lower, than the expression of the same chemokine/cytokine in a control sample, while the expression of a second chemokine/cytokine may be 1.5× lower, or about 1.5× lower, than the expression of the same chemokine/cytokine in a control sample. As discussed above, altered expression includes both higher and lower expression of the chemokine/cytokine, compared to a control level.

### EXAMPLES

### Example 1: A Placebo-Controlled Study to Evaluate the Safety and Efficacy of a BCG Vaccine in Treating Attention-Deficit/Hyperactivity Disorder (ADHD)

The primary objective of this study will be to evaluate the efficacy of a BCG vaccine for boosting the immune system of patients suffering from ADHD. Secondary objectives will be to evaluate the safety and tolerability of both of the BCG vaccine as well as investigate local injection site reactions.

Study Patients and Dosing Schedule

The study will be in the form of a prospective, randomized, double-blind, placebo-controlled, parallel-group study using a BCG vaccine. Male and female patients (over 18 years) who have been previously diagnosed with ADHD are included in this trial. The main exclusion criteria will include pregnant and lactating woman, patients suffering from other inflammatory rheumatological diseases (such as rheumatoid arthritis or collagenoses), severe neuropathies, clinically manifest endocrinopathies, bone diseases, severe cardial, renal or hepatic impairment and acute or chronic infections.

50 Patients will be randomly assigned to one of two study cohorts: placebo (25 patients; cohort 1) or BCG vaccine (25 patients; cohort 2). The BCG used will be Tokyo BCG (JBL, Tokyo, Japan). The duration of treatment will be four weeks over which each patient will receive (2) intradermal (ID) injections. Each dose will be administered in each patient from one of the two cohorts into the skin overlying the deltoid muscle with the arm alternated for each dose. Prior to commencement of the study, each patient in each cohort will receive a placebo injection of borate saline solution (day −3) to provide an intra-patient placebo control and to allow the patient to practice completion of the diary and assess whether patients are capable of measuring their own in injection site reactions accurately. Patients who are willing and able to proceed with the study will be injected with a single dose level of placebo (borate saline-solution, cohort 1) or BCG (cohort 2) on (2) subsequent occasions. Doses of placebo or BCG will be administered over a 4-week period on days 0 and 28 (with up to 2 days variation in the dosing interval). For cohort 1, a standard volume of 0.1 ml of borate saline-solution will be injected. For cohort 2, a standard volume of 0.1 ml of a suspension containing BCG at the concentration of 1.8 to 3.9×10⁶ cfu will be injected. All injections will be administered using a Biojector® B2000 device for intradermal (ID) administration.

Before, on each dosing day and at the end of the treatment phase (i.e., at screening and at days 3, 14, 28 and 42), routine safety assessments will be performed using physical examinations, urinalysis, electrocardiograms and hematological and biochemical blood tests. Further, at days 0, 3, 14, 28 and 42, local tolerability will be assessed using standardized techniques (measurements at injection site) by a study physician or research nurse and the intensity of each injection site reaction will be scored with reference to a Vaccine Toxicology Rating Scale (available at Annals of Oncology online). Additionally, at screening and at days 3, 14, 28 and 42, a physical examination, a pain assessment and full medical write-up will be performed in order to assess physical signs and symptoms of ADHD as well as the biochemical effects of the vaccination protocol, respectively.

In order to document daily the intensity of pain, adverse events and concomitant medications, patients will use a standardized diary and will record daily the parameters mentioned. In addition, changes in functional symptoms will be documented at start of treatment, as well as on day 3, day 14, day 28 and at the end of treatment (day 42). Adverse events will be assessed during the active treatment period.

To evaluate pain, the pain score, a visual analogue scale and clinical examination of tender points will be used. The pain score ranges from 0 to 120, measuring the pain intensity in 24 body regions applied to the following rating scale: 0=no pain, 1=mild pain, 2=moderate pain, 3=moderately severe pain, 4=severe pain, 5=most ever pain. The assessment of each body region will be done by the patients themselves; the total score will be calculated as the sum of the regional scores.

The visual analogue scale is in the form of 100-mm-line oriented horizontally with one end=0, indicating “no pain” and the other end=100, indicating “worst pain”. The patients are asked to place a mark corresponding to their perception of their present pain intensity.

In addition to the documented effects during the active treatment phase, a follow-up of the patients will be performed for six months in order to evaluate the duration of the clinical response.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

All publications, patents and patent applications herein are incorporated by reference to the same extent as if each individual publication or patent application was specifically and individually indicated to be incorporated by reference. It is not an admission that any of the information provided herein is prior art or relevant to the presently claimed inventions, or that any publication specifically or implicitly referenced is prior art.

While the invention has been described in connection with specific embodiments thereof, the foregoing description has been given for clearness of understanding only and no unnecessary limitations should be understood therefrom. It will be understood that the description is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. Those skilled in the art will recognize, or be able to ascertain using no more than routine experimentation, many equivalents to the specific embodiments of the invention described herein. Such equivalents are intended to be encompassed by the following claims.

### Further Numbered Embodiments of the Disclosure

Other subject matter contemplated by the present disclosure is set out in the following numbered embodiments:

1. A method of treating attention-deficit/hyperactivity disorder (ADHD) and the symptoms associated with such a disorder in a subject, comprising administering a therapeutically effective amount of a composition comprising an isolated Mycobacterium to the subject, wherein the subject suffers from or is suspected of suffering from attention-deficit/hyperactivity disorder (ADHD).

2. The method of embodiment 1, further comprising diagnosing the subject with ADHD prior to the administration of the therapeutically effective amount of the composition comprising an isolated Mycobacterium.

3. The method of embodiment 2, wherein the diagnosing comprises testing of the subject for an allergic disorder.

4. The method of embodiment 3, wherein the allergic disorder is selected from asthma, eczema, rhinitis, urticaria or any combination thereof.

5. The method of embodiment 2, wherein the diagnosing comprises conducting an assessment of immunologic traits associated with ADHD, an assessment of behaviors associated with ADHD or any combination thereof.

6. The method of embodiment 5, wherein the immunologic traits are alterations in chemokine and/or cytokine production, wherein the alterations are an increase in production of one or more chemokines or cytokines, a decrease in production in one or more chemokines or cytokines or any combination thereof.

7. The method of embodiment 5 or 6, wherein the immunologic traits associated with ADHD are occurrence of allergic disorders, atopy, hypersensitivity to allergens or any combination thereof.

8. The method of embodiment 7, wherein the allergic disorders are asthma, eczema, rhinitis, urticaria, or any combination thereof.

9. The method of embodiment 2, wherein the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample.

10. The method of embodiment 9, wherein the blood sample obtained from the subject is peripheral blood mononuclear cells (PBMCs) isolated from plasma of the blood sample obtained from the subject.

11. The method of embodiment 10, wherein the PBMCs are stimulated with one or more mitogens prior to determination of the expression levels of the at least four cytokines.

12. The method of embodiment 9, wherein the determined expression level is a protein expression level, wherein the protein expression level is determined using an antibody bead-based capture assay that comprises beads derivatized with antibodies specific to the at least four cytokines.

13. The method of any one of the above embodiments, wherein the Mycobacterium comprises a whole cell Mycobacterium.

14. The method of any one of the above embodiments, wherein the Mycobacterium comprises a live-attenuated Mycobacterium.

15. The method of any one of embodiments 1-13, wherein the Mycobacterium comprises a heat-killed Mycobacterium.

16. The method of any one of the above embodiments, wherein the Mycobacterium is M. bovis.

17. The method of any one of the above embodiments, wherein the Mycobacterium is a Bacille Calmette-Guerin (BCG) strain.

18. The method of embodiment 15, wherein the heat-killed Mycobacterium is a non-pathogenic Mycobacterium.

19. The method of embodiment 18, wherein the non-pathogenic Mycobacterium is selected from M. vaccae, M. obuense, M. parafortuitum, M. aurum, M. indicus pranii, and combinations thereof.

20. The method of embodiment 19, wherein the Mycobacterium is selected from M. vaccae or M. obuense.

21. The method of any one of embodiments 18-20, wherein the Mycobacterium is a rough variant.

22. The method of any one of the above embodiments, wherein the Mycobacterium is in the form of a vaccine composition optionally comprising an adjuvant.

23. The method of embodiment 22, wherein the vaccine composition induces one or more epigenetic changes in the genome of the individual.

24. The method of any one of the above embodiments, wherein the Mycobacterium is administered in repeat doses.

25. The method of any one of the above embodiments, wherein the Mycobacterium is administered in a unit dose comprising an effective amount of Mycobacterium from 107 to 109 cells.

26. The method of any one of embodiments 1-24, wherein the Mycobacterium is administered in a unit dose comprising an effective amount of Mycobacterium from 0.1 mg to 1 mg.

27. The method of any one of the above embodiments, wherein the Mycobacterium is formulated for administration via the parenteral, oral, sublingual, nasal or pulmonary route.

28. The method of embodiment 27, wherein the Mycobacterium is formulated for administration via the oral route.

29. The method of embodiment 27, wherein the parenteral route is selected from subcutaneous, intradermal, subdermal, intraperitoneal, intravenous, or intravesicular injection.

30. The method of any one of the above embodiments, wherein the administration of the composition comprising the isolated Mycobacterium prevents, reduces or alleviates at least one sign or symptom of ADHD, wherein the sign or symptom is selected from inattention, impulsivity, hyperactivity or a combination thereof.

31. The method of any one of the above embodiments, wherein the administration of the composition comprising the isolated Mycobacterium elevates or increases immune system activity of the subject.

32. The method of embodiment 31, wherein the elevation or increase in immune system function is evidenced by the production of TH1 cytokines, upregulation of granzyme B or both.

33. A method of elevating or increasing a subject's immune system function comprising administering a therapeutically effective amount of a Mycobacterium vaccine to the subject, wherein the subject suffers from or is suspected of suffering from attention-deficit/hyperactivity disorder (ADHD).

34. The method of embodiment 33, further comprising diagnosing the subject with ADHD prior to the administration of the therapeutically effective amount of the composition comprising an isolated Mycobacterium.

35. The method of embodiment 34, wherein the diagnosing comprises testing of the subject for an allergic disorder.

36. The method of embodiment 35, wherein the allergic disorder is selected from asthma, eczema, rhinitis, urticaria or any combination thereof.

37. The method of embodiment 34, wherein the diagnosing comprises conducting an assessment of immunologic traits associated with ADHD, an assessment of behaviors associated with ADHD or any combination thereof.

38. The method of embodiment 37, wherein the immunologic traits are alterations in chemokine and/or cytokine production, wherein the alterations are an increase in production of one or more chemokines or cytokines, a decrease in production in one or more chemokines or cytokines or any combination thereof.

39. The method of embodiment 37 or 38, wherein the immunologic traits associated with ADHD are occurrence of allergic disorders, atopy, hypersensitivity to allergens or any combination thereof.

40. The method of embodiment 39, wherein the allergic disorders are asthma, eczema, rhinitis, urticaria, or any combination thereof.

41. The method of embodiment 34, wherein the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample.

42. The method of embodiment 41, wherein the blood sample obtained from the subject is peripheral blood mononuclear cells (PBMCs) isolated from plasma of the blood sample obtained from the subject.

43. The method of embodiment 42, wherein the PBMCs are stimulated with one or more mitogens prior to determination of the expression levels of the at least four cytokines.

44. The method of embodiment 41, wherein the determined expression level is a protein expression level, wherein the protein expression level is determined using an antibody bead-based capture assay that comprises beads derivatized with antibodies specific to the at least four cytokines.

45. The method of any one of embodiments 33-44, wherein the elevation or increase in immune system function is evidenced by the production of TH1 cytokines, upregulation of granzyme B or both.

46. The method of any one of embodiments 33-45, wherein the elevation or increase in immune system function is evidenced by a reduction or alleviation of at least one sign or symptom of attention-deficit/hyperactivity disorder (ADHD).

47. The method of embodiment 46, wherein the sign or symptom is selected from inattention, impulsivity, hyperactivity or a combination thereof.

48. The method of any one of embodiments 33-47, the Mycobacterium vaccine composition induces one or more epigenetic changes in the genome of the individual.

49. The method of any one of embodiments 33-48, wherein the Mycobacterium vaccine comprises a live-attenuated Mycobacterium.

50. The method of any one of embodiments 33-48, wherein the Mycobacterium vaccine comprises a heat-killed Mycobacterium.

51. The method of any one of embodiments 33-50, wherein the Mycobacterium is M. bovis.

52. The method of any one of embodiments 33-51, wherein the Mycobacterium is a Bacille Calmette-Guerin (BCG) strain.

53. The method of embodiment 50, wherein the heat-killed Mycobacterium is a non-pathogenic Mycobacterium.

54. The method of embodiment 53, wherein the non-pathogenic Mycobacterium is selected from M. vaccae, M. obuense, M. parafortuitum, M. aurum, M. indicus pranii, and combinations thereof.

55. The method of embodiment 54, wherein the Mycobacterium is selected from M. vaccae or M. obuense.

56. The method of any one of embodiments 53-55, wherein the Mycobacterium is a rough variant.

57. The method of any one of embodiments 33-56, wherein the vaccine further comprises an adjuvant.

58. The method of any one of embodiments 33-57, wherein the Mycobacterium vaccine is administered in repeat doses.

59. The method of any one of embodiments 33-58, wherein the Mycobacterium vaccine is administered in a unit dose comprising an effective amount of Mycobacterium from 107 to 109 cells.

60. The method of any one of embodiments 33-57, wherein the Mycobacterium vaccine is administered in a unit dose comprising an effective amount of Mycobacterium from 0.1 mg to 1 mg.

61. The method of any one of embodiments 33-60, wherein the Mycobacterium vaccine is formulated for administration via the parenteral, oral, sublingual, nasal or pulmonary route.

62. The method of embodiment 61, wherein the Mycobacterium vaccine is formulated for administration via the oral route.

63. The method of embodiment 61, wherein the parenteral route is selected from subcutaneous, intradermal, subdermal, intraperitoneal, intravenous, or intravesicular injection.

64. A method of treating attention-deficit/hyperactivity disorder (ADHD) and the symptoms associated with such a disorder in a subject, comprising diagnosing a subject with ADHD; and administering a therapeutically effective amount of a composition comprising an isolated Mycobacterium to the subject diagnosed with ADHD.

65. The method of embodiment 64, wherein the diagnosing comprises testing of the subject for an allergic disorder.

66. The method of embodiment 65, wherein the allergic disorder is selected from asthma, eczema, rhinitis, urticaria or any combination thereof.

67. The method of embodiment 64, wherein the diagnosing comprises conducting an assessment of immunologic traits associated with ADHD, an assessment of behaviors associated with ADHD or any combination thereof.

68. The method of embodiment 67, wherein the immunologic traits are alterations in chemokine and/or cytokine production, wherein the alterations are an increase in production of one or more chemokines or cytokines, a decrease in production in one or more chemokines or cytokines or any combination thereof.

69. The method of embodiment 67 or 68, wherein the immunologic traits associated with ADHD are occurrence of allergic disorders, atopy, hypersensitivity to allergens or any combination thereof.

70. The method of embodiment 69, wherein the allergic disorders are asthma, eczema, rhinitis, urticaria, or any combination thereof.

71. The method of embodiment 64, wherein the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample.

72. The method of embodiment 71, wherein the blood sample obtained from the subject is peripheral blood mononuclear cells (PBMCs) isolated from plasma of the blood sample obtained from the subject.

73. The method of embodiment 72, wherein the PBMCs are stimulated with one or more mitogens prior to determination of the expression levels of the at least four cytokines.

74. The method of embodiment 71, wherein the determined expression level is a protein expression level, wherein the protein expression level is determined using an antibody bead-based capture assay that comprises beads derivatized with antibodies specific to the at least four cytokines.

75. The method of any one of embodiments 64-74, wherein the Mycobacterium comprises a whole cell Mycobacterium.

76. The method of any one of embodiments 64-75, wherein the Mycobacterium comprises a live-attenuated Mycobacterium.

77. The method of any one of embodiments 64-75, wherein the Mycobacterium comprises a heat-killed Mycobacterium.

78. The method of any one of embodiments 64-77, wherein the Mycobacterium is M. bovis.

79. The method of any one of embodiments 64-78, wherein the Mycobacterium is a Bacille Calmette-Guerin (BCG) strain.

80. The method of embodiment 77, wherein the heat-killed Mycobacterium is a non-pathogenic Mycobacterium. 81. The method of embodiment 80, wherein the non-pathogenic Mycobacterium is selected from M. vaccae, M. obuense, M. parafortuitum, M. aurum, M. indicus pranii, and combinations thereof.

82. The method of embodiment 81, wherein the Mycobacterium is selected from M. vaccae or M. obuense.

83. The method of any one of embodiments 80-82, wherein the Mycobacterium is a rough variant.

84. The method of any one of embodiments 64-83, wherein the Mycobacterium is in the form of a vaccine composition optionally comprising an adjuvant.

85. The method of embodiment 84, wherein the vaccine composition induces one or more epigenetic changes in the genome of the individual.

86. The method of any one of embodiments 64-85, wherein the Mycobacterium is administered in repeat doses.

87. The method of any one of embodiments 64-86, wherein the Mycobacterium is administered in a unit dose comprising an effective amount of Mycobacterium from 107 to 109 cells.

88. The method of any one of embodiments 64-86, wherein the Mycobacterium is administered in a unit dose comprising an effective amount of Mycobacterium from 0.1 mg to 1 mg.

89. The method of any one of embodiments 64-88, wherein the Mycobacterium is formulated for administration via the parenteral, oral, sublingual, nasal or pulmonary route.

90. The method of embodiment 89, wherein the Mycobacterium is formulated for administration via the oral route.

91. The method of embodiment 89, wherein the parenteral route is selected from subcutaneous, intradermal, subdermal, intraperitoneal, intravenous, or intravesicular injection.

92. The method of any one of the embodiments 64-91, wherein the administration of the composition comprising the isolated Mycobacterium prevents, reduces or alleviates at least one sign or symptom of ADHD, wherein the sign or symptom is selected from inattention, impulsivity, hyperactivity or a combination thereof.

93. The method of any one of embodiments 64-92, wherein the administration of the composition comprising the isolated Mycobacterium elevates or increases immune system activity of the subject.

94. The method of embodiment 93, wherein the elevation or increase in immune system function is evidenced by the production of TH1 cytokines, upregulation of granzyme B or both.

1. A method of treating attention-deficit/hyperactivity disorder (ADHD) and the symptoms associated with such a disorder in a subject, comprising administering a therapeutically effective amount of a composition comprising an isolated Mycobacterium to the subject, wherein the subject suffers from or is suspected of suffering from attention-deficit/hyperactivity disorder (ADHD).

2. The method of claim 1, further comprising diagnosing the subject with ADHD prior to the administration of the therapeutically effective amount of the composition comprising an isolated Mycobacterium.

3. The method of claim 2, wherein the diagnosing comprises testing of the subject for an allergic disorder or conducting an assessment of immunologic traits associated with ADHD, an assessment of behaviors associated with ADHD or any combination thereof.

4.-5. (canceled)

6. The method of claim 3, wherein the immunologic traits are alterations in chemokine and/or cytokine production, wherein the alterations are an increase in production of one or more chemokines or cytokines, a decrease in production in one or more chemokines or cytokines or any combination thereof.

7. The method of claim 3, wherein the immunologic traits associated with ADHD are occurrence of allergic disorders, atopy, hypersensitivity to allergens or any combination thereof.

8. (canceled)

9. The method of claim 2, wherein the diagnosing comprises: determining expression levels of at least four cytokines selected from the group consisting of IL-2, IL-4, IL5, IL6, IL8, IL 10, IFN-γ, RANTES, MCP-1, MIP-α and MIP-β in a blood sample obtained from the subject; comparing the detected levels of expression to the expression of the at least four cytokines in a control sample; and identifying the subject as suffering from ADHD if the at least four cytokines have an altered expression in the blood sample as compared to the expression levels of the at least four cytokines in the control sample.

10. The method of claim 9, wherein the blood sample obtained from the subject is peripheral blood mononuclear cells (PBMCs) isolated from plasma of the blood sample obtained from the subject.

11. The method of claim 10, wherein the PBMCs are stimulated with one or more mitogens prior to determination of the expression levels of the at least four cytokines.

12. The method of claim 9, wherein the determined expression level is a protein expression level, wherein the protein expression level is determined using an antibody bead-based capture assay that comprises beads derivatized with antibodies specific to the at least four cytokines.

13.-15. (canceled)

16. The method of claim 1, wherein the Mycobacterium is selected from M. bovis, M. vaccae, M. obuense, M. parafortuitum, M. aurum, M. indicus pranii, and combinations thereof.

17. The method of claim 1, wherein the Mycobacterium is a Bacille Calmette-Guerin (BCG) strain.

18.-29. (canceled)

30. The method of claim 1, wherein the administration of the composition comprising the isolated Mycobacterium prevents, reduces or alleviates at least one sign or symptom of ADHD, wherein the sign or symptom is selected from inattention, impulsivity, hyperactivity or a combination thereof.

31. The method of claim 1, wherein the administration of the composition comprising the isolated Mycobacterium elevates or increases immune system activity of the subject.

32. The method of claim 31, wherein the elevation or increase in immune system function is evidenced by the production of T_{H}1 cytokines, upregulation of granzyme B or both.

33. A method of elevating or increasing a subject's immune system function comprising administering a therapeutically effective amount of a Mycobacterium vaccine to the subject, wherein the subject suffers from or is suspected of suffering from attention-deficit/hyperactivity disorder (ADHD).

34.-44. (canceled)

45. The method of claim 33, wherein the elevation or increase in immune system function is evidenced by the production of T_{H}1 cytokines, upregulation of granzyme B or both.

46. The method of claim 33, wherein the elevation or increase in immune system function is evidenced by a reduction or alleviation of at least one sign or symptom of attention-deficit/hyperactivity disorder (ADHD).

47. (canceled)

48. The method of claim 33, the Mycobacterium vaccine composition induces one or more epigenetic changes in the genome of the individual.

49.-50. (canceled)

51. The method of claim 33, wherein the Mycobacterium is selected from M. bovis, M. vaccae, M. obuense, M. parafortuitum, M. aurum, M. indicus pranii, and combinations thereof.

52. The method of claim 33, wherein the Mycobacterium is a Bacille Calmette-Guerin (BCG) strain.

53.-94. (canceled)
